(19) United States
(12) Patent Application Publication
(10) Pub. No.: US 2026/0034188 A1
(43) Pub. Date: Feb. 5, 2026

(54) METHOD OF PREPARING ALOESWOOD WATER CONTAINING ALOESWOOD WITH ATOPIC DERMATITIS AND ACNE AMELIORATING EFFECTS

(71) Applicant: Jong Moon JUNG, Daegu-si (KR)

(72) Inventor: Jong Moon JUNG, Daegu-si (KR)

(21) Appl. No.: 18/904,079

(22) Filed: Oct. 2, 2024

(30) Foreign Application Priority Data
Jul. 30, 2024 (KR) 10-2024-0100682

(51) Int. Cl. A61K 36/83

(52) U.S. Cl. CPC A61K 36/83; A61K 2236/13; A61K 2236/15; A61K 2236/17; A61K 2236/31

(57) ABSTRACT

The present invention provides a method of preparing aloeswood water, including: a material preparation step in which materials are prepared by washing each of aloeswood, Phellinus linteus, Houttuynia cordata Thunb., Saururus chinensis, green tea or puer tea, Stachys sieboldii, Lycopus lucidus Turcz., Agrimonia pilosa, and Panax notoginsengs, and then drying in the shade; a material pulverization step in which each of those, dried through the material preparation step, is pulverized; a boiling step in which, after the material pulverization step is completed, the powders thereof are mixed to prepare a material mixture, water is mixed, and the resulting mixture is heated and boiled; an infusing step in which, after the boiling step is completed, infusing is performed by cooling the boiled water; and a reduced pressure distillation step in which, after the infusing step is completed, the infused water is distilled under reduced pressure.

### CROSS-REFERENCE TO RELATED APPLICATIONS

This U.S. non-provisional patent application claims priority under 35 U.S.C. § 119 of Korean Patent Application No. 10-2024-0100682 filed on Jul. 30, 2024, the entirety of which is hereby incorporated by reference.

### BACKGROUND OF THE INVENTION

### Field of the Invention

The present invention relates to a method of preparing aloeswood water containing aloeswood, and more specifically, to a method of preparing aloeswood water containing aloeswood having atopic dermatitis and acne ameliorating effects, which may provide atopic dermatitis and acne ameliorating effects.

### Description of the Related Art

Aloeswood refers to a hardened resin of an evergreen tall tree of the family Thymelaeaceae.

This aloeswood is made by burying juniper or oak trees at the confluence of seawater and mountain stream water, and the various mineral components contained in the seawater and mountain stream water seep into the trees over a long period of time. Unlike general wood, aloeswood refers to hard wood that sinks in water.

However, aloeswood, which is formed by the hardening of resin in the aloeswood tree, is rare and very expensive worldwide, so it is traded in units of less than a gram, like gold or jewels. In addition, producing aloeswood by burying juniper trees takes a long time, so there is an inherent problem in that it is difficult to easily purchase and use for various purposes.

This aloeswood has been used as a medicinal herb or fragrance since long ago, and it is recorded in the Ben Cao Gang Mu (Compendium of Materia Medica) that aloeswood is effective in sedation, stomach strengthening, energy circulation and ameliorating indigestion, loss of appetite, vomiting, bronchial asthma, premature ejaculation, and lack of energy.

As the pharmacological activities of aloeswood, a central nervous system suppressing effect and a cathartic effect are reported, and antibacterial, anticancer, anti-inflammatory, and anti-allergic effects are also reported. In addition, aloeswood contains active ingredients such as agarospirol, which relaxes the central nervous system and clears the mind, agarofuran, which stabilizes the central nervous system, terpineol, which has an antispasmodic effect, and dehydrofukinone, which stabilizes the central nervous system. Therefore, aloeswood has the effect of stabilizing the brain of the central nervous system, relaxing the central nervous system, and clearing the brain, thereby improving memory and/or cognitive abilities.

Meanwhile, regarding the related arts on aloeswood, Korean Patent 10-1044641 relates to a method of producing aloeswood, the method including: cutting juniper or oak wood according to the purpose; drying the wood cut according to the purpose in a fan drying chamber having an internal temperature of 90° C. to 95° C. for 2 hours 30 minutes to 3hours 30 minutes; infiltrating mixed water obtained by mixing concentrated deep sea water and stream water at a ratio of 3:1 to the inner surface of the dried wood in a drying chamber under high pressure; drying the wood infiltrated with the mixed water in a fan drying chamber; repeatedly performing the high-pressure infiltrating operation and the drying operation; and inspecting the density and fragrance of the wood completely produced by repeatedly performing the high-pressure infiltrating operation with the mixed water and the drying operation, and separating the wood satisfying conditions. The technology is for producing aloeswood with high fragrance and density in a short period of time by cutting juniper or oak wood into various sizes. The technology has the effect that the aloeswood produced in this manner can be supplied and used not only as a high-quality incense but also in various fields across industries, such as carving materials, vehicle interior decoration materials, and mobile phone exterior materials.

In addition, Korean Patent No. 10-0931880 relates to an oriental medicine drink containing aloeswood, Sasa borealis, Polygala tenuifolia, and Zizyphus jujuba, and a method of preparing the same.

The technology is directed to a method of preparing an oriental medicine drink having the effect of reducing the total cholesterol concentration without side effects and maintaining the cardiac vessels to be clean, thereby preventing heart attacks caused by myocardial infarction, and the effect of ameliorating depression by resolving insomnia, more specifically, the technology relates to a method of method of preparing an oriental medicine drink containing aloeswood, Sasa borealis, Polygala tenuifolia, and Zizyphus jujuba, the method including: mixing 100 to 150 parts by weight of Sasa borealis, 150to 200 parts by weight of Polygala tenuifolia, and 100 to 150 parts by weight of Zizyphus jujuba based on 100 parts by weight of aloeswood; mixing the resulting mixture with water in an amount of 1.5 to 6 times by weight of the total weight of the mixture in a container; and distilling by heating the container and passing the steam discharged from a steam outlet through a cooler to prepare a distilled solution.

### RELATED ARTS

### Patent Documents

(Patent Document 1) Patent Document 1. Korean Patent Publication 10-1044641 (registered on Jun. 21, 2011)
(Patent Document 2) Patent Document 2. Korean Patent Publication 10-0931880 (registered on Dec. 7, 2009)

### SUMMARY OF THE INVENTION

The present invention has been created to overcome the above-described problems, and provides a method of preparing aloeswood water prepared by dispersing active ingredients of aloeswood in water, and an easily drinkable aloeswood water prepared using the method, which can provide atopic dermatitis and acne ameliorating effects.

The present invention provides a method of preparing aloeswood water, including: a material preparation step in which materials are prepared by washing each of aloeswood, mulberry Phellinus linteus, Houttuynia cordata Thunb., Saururus chinensis, green tea or puer tea, Stachys sieboldii, Lycopus lucidus Turcz., Agrimonia pilosa, and Panax notoginsengs, and then drying in the shade; a material pulverization step in which each of the aloeswood, mulberry Phellinus linteus, Houttuynia cordata Thunb., Saururus chinensis, green tea or puer tea, Stachys sieboldii, Lycopus lucidus Turcz., Agrimonia pilosa, and Panax notoginsengs, dried through the material preparation step, is pulverized; a boiling step in which, after the material pulverization step is completed, aloeswood powder, mulberry Phellinus linteus powder, Houttuynia cordata Thunb. powder, Saururus chinensis powder, green tea powder or puer tea powder, Stachys sieboldii powder, Lycopus lucidus Turcz. powder, Agrimonia pilosa powder, and Panax notoginsengs powder are mixed at a weight ratio of 3:1.3 to 1.7:1.3 to 1.7:1.1 to 1.5:0.8 to 1.2:0.3 to 0.7:0.5 to 0.9:0.1 to 0.5:0.1 to 0.4 to prepare a material mixture, water in an amount of 9,000 to 11,000 parts by weight based on 100 part by weight of the material mixture is mixed, and the resulting mixture is heated and boiled in a temperature range of 80 to 100° C. for 22 to 26 hours; an infusing step in which, after the boiling step is completed, infusing is performed by keeping the boiled water in a temperature range of 15 to 25° C. for 22 to 26 hours; and a reduced pressure distillation step in which, after the infusing step is completed, the infused water is distilled under reduced pressure.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIGS. 1A, 1B, and 1C are photographs of drying an example of materials according to the present invention;

FIGS. 2A and 2B are photographs of powder showing an example of pulverizing materials according to the present invention;

FIG. 3 is a photograph showing an example of heating and infusing powder materials according to the present invention; and

FIG. 4 shows photographs illustrating an example of distillation under reduced pressure after an infusing step according to the present invention.

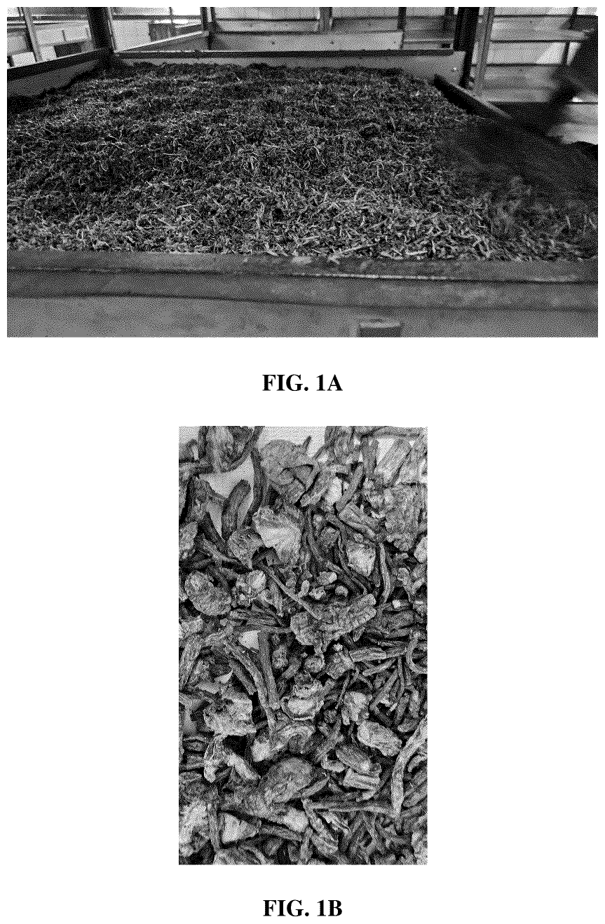

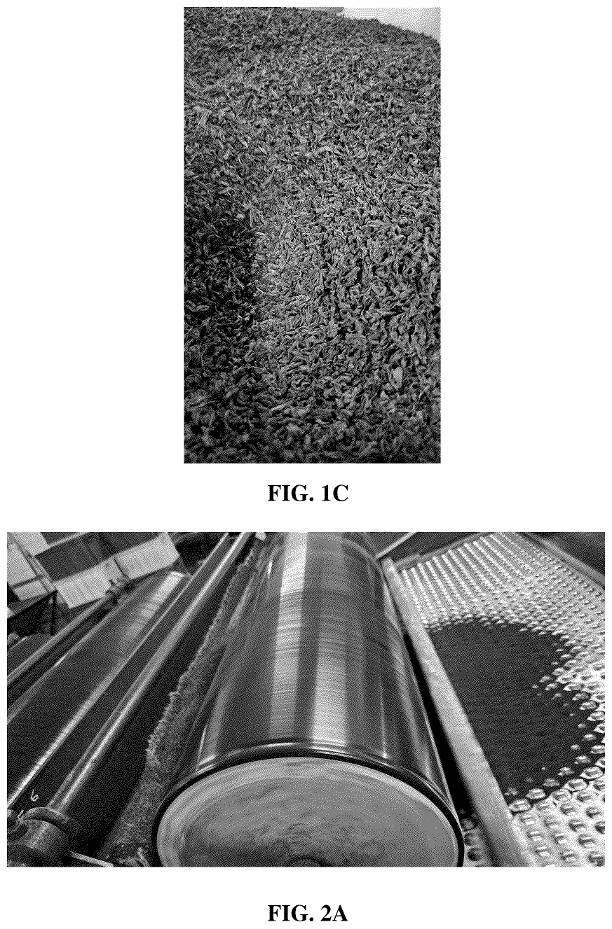

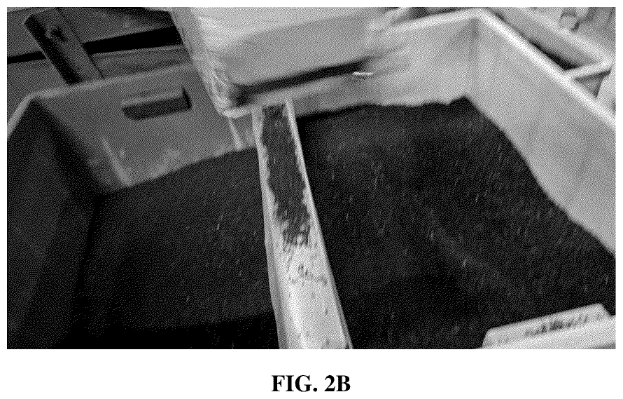

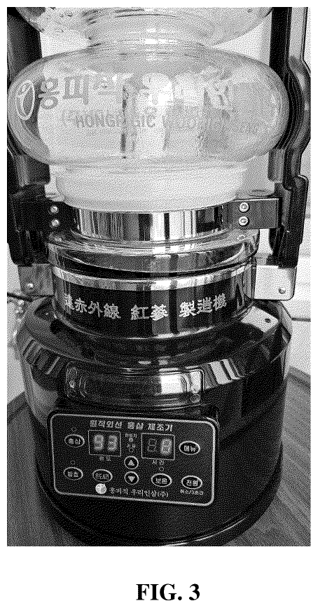

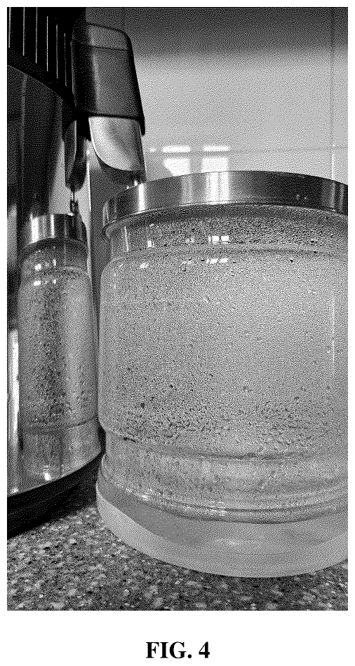

### DETAILED DESCRIPTION OF THE EMBODIMENTS

Hereinafter, the present invention will be described in detail.

The present invention provides a method of preparing aloeswood water, including: a material preparation step in which materials are prepared by washing each of aloeswood, mulberry Phellinus linteus, Houttuynia cordata Thunb., Saururus chinensis, green tea or puer tea, Stachys sieboldii, Lycopus lucidus Turcz., Agrimonia pilosa, and Panax notoginsengs, and then drying in the shade; a material pulverization step in which each of the aloeswood, mulberry Phellinus linteus, Houttuynia cordata Thunb., Saururus chinensis, green tea or puer tea, Stachys sieboldii, Lycopus lucidus Turcz., Agrimonia pilosa, and Panax notoginsengs, dried through the material preparation step, is pulverized; a boiling step in which, after the material pulverization step is completed, aloeswood powder, mulberry Phellinus linteus powder, Houttuynia cordata Thunb. powder, Saururus chinensis powder, green tea powder or puer tea powder, Stachys sieboldii powder, Lycopus lucidus Turcz. powder, Agrimonia pilosa powder, and Panax notoginsengs powder are mixed at a weight ratio of 3:1.3 to 1.7:1.3 to 1.7:1.1 to 1.5:0.8 to 1.2:0.3 to 0.7:0.5 to 0.9:0.1 to 0.5:0.1 to 0.4 to prepare a material mixture, water in an amount of 9,000 to 11,000 parts by weight based on 100 part by weight of the material mixture is mixed, and the resulting mixture is heated and boiled in a temperature range of 80 to 100° C. for 22 to 26 hours; an infusing step in which, after the boiling step is completed, infusing is performed by keeping the boiled water in a temperature range of 15 to 25° C. for 22 to 26 hours; and a reduced pressure distillation step in which, after the infusing step is completed, the infused water is distilled under reduced pressure.

In the method of preparing aloeswood water according to the present invention, specifically, in the method of preparing aloeswood water containing aloeswood, and more specifically, in the method of preparing aloeswood water containing aloeswood with atopic dermatitis and acne ameliorating effects, the materials used include aloeswood, mulberry Phellinus linteus, Houttuynia cordata Thunb., Saururus chinensis, green tea or puer tea, Stachys sieboldii, Lycopus lucidus Turcz., Agrimonia pilosa, and/or Panax notoginsengs.

Here, it is preferable to use the leaves of the aloeswood, mulberry Phellinus linteus, Houttuynia cordata Thunb., Saururus chinensis, green tea or puer tea, Stachys sieboldii, Lycopus lucidus Turcz., Agrimonia pilosa, and/or Panax notoginsengs, but the present invention is not limited thereto, and seeds, roots, stems, and/or fruits and the like may also be used.

In particular, the materials used in the method of preparing aloeswood water according to the present invention, such as aloeswood, mulberry Phellinus linteus, Houttuynia cordata Thunb., Saururus chinensis, green tea or puer tea, Stachys sieboldii, Lycopus lucidus Turcz., Agrimonia pilosa, and/or Panax notoginsengs, have been found through various curing cases to have antiviral, anti-allergic, anti-inflammatory, antispasmodic, and analgesic effects and help promote physical and mental health, confirming that they have excellent anti-atopic dermatitis and anti-acne effects.

The aloeswood according to the present invention is formed by hardening the resin in the aloeswood tree, and any aloeswood commonly used in the art that may be consumed by eating and drinking may be used.

In particular, the aloeswood water prepared by infusing aloeswood according to the present invention has excellent fragrance, and is rich in monoterpene, terpene, and sesquiterpene-based substances, so it has anti-allergic, anti-spasmodic, analgesic, and sedative effects, helps physical recovery and health promotion, and provides pharmacological properties such as anticancer, anti-inflammatory, and antiviral effects, and thus is effective in ameliorating atopic dermatitis and acne.

The mulberry Phellinus linteus according to the present invention is a perennial mushroom of the family Basidiomycete, a yellow mushroom that grows on the rotting stumps of mulberry trees, and it is a medicinal mushroom with effects such as strengthening the immune system, preventing aging, and beautifying the skin. In addition, it is also known that effects such as reducing cholesterol, controlling blood sugar, and controlling blood pressure may be expected therefrom.

Here, the above Phellinus linteus is a mushroom of the family Phellinus linteus in the order Amanitas in the phylum Basidiomycetes in the plant kingdom, and it is a perennial wood-rotting fungus that grows on mulberry trees and the like.

In particular, the Phellinus linteus initially looks like a lump of mud, but after it fully grows, it resembles a tongue sticking out of a tree stump, so it is also called ‘tree tongue’. In Donguibogam (Principles and Practice of Eastern Medicine), it is recorded in the section of decoction under the name Sangmoki, and it grows natively in Korea, Japan, Australia, and North America.

Furthermore, in the Ben Cao Gang Mu (Compendium of Materia Medica), it is written that the Phellinus linteus “is cold, non-toxic, and mild, controls toxins, is good for skin beauty, women's hematuria, abdominal pain, women's leukorrhea, women's genital pain, and the five internal organs, and is good for stamina.” The Phellinus linteus contains high-quality ingredients such as protein polysaccharides, amino acids, hydrocarbons, fatty acids, enzymes, K, Ca, and vitamin B, and the beta-glucan contained in Phellinus linteus strengthens immune cells and improves immune functions.

Among different types of Phellinus linteus, the mulberry Phellinus linteus, which grows on mulberry trees, has the effects of enhancing immunity, detoxifying various toxins, removing blemishes and freckles, and ameliorating and treating atopic dermatitis and acne. The mulberry Phellinus linteus is mainly harvested in the fall, and Gangwon-do is the main producing area in Korea. It is eaten raw, dried and made into tea, or used as a food ingredient.

Houttuynia cordata Thunb. according to the present invention is a perennial herb belonging to the family Trifoliaceae, and is the whole plant with the roots. The whole plant is pulled out with the roots in summer and fall and used. The root contains essential oil, which contains antibacterial components such as decanoyl acetaldehyde, methyl-n-nonyl-ketone, α-pinene, linalool, and d-limonen, and, it also contains quercitrin (flavonoid), cordarine, alkaloids, and the like.

Quercitrin (flavonoid) contained in Houttuynia cordata Thunb. has anticancer and detoxifying effects, reduces the toxicity of heavy metals such as cadmium, ameliorates leukorrhea in women, and is excellent in eliminating toxins from the body such as acne.

The Saururus chinensis according to the present invention is a type of perennial herb of the family Saururus of the order Piperales, and its rhizome is white and grows sideways in the mud. The height of the stem is 50 to 100 cm, and white flowers bloom from June to August. It mainly grows in wetlands, and is called ‘plant of three whites’ because its roots, leaves, and flowers are white. It is known to be effective for jaundice, hepatitis, and the like, and is found in Korea, Japan, China, and the like.

The Saururus chinensis purifies the blood and clean the blood vessel walls, so they are effective in preventing and treating heart diseases, high blood pressure, and cardiac diseases such as angina pectoris, and it enhances the liver's detoxification function to improve liver functions, and strengthens the kidney function to treat renal inflammation. In addition, it contains quercitrin, quercetin, and isoflavones, so it may prevent cancer or lifestyle diseases while removing tumors in the body, and it has the effect of removing lipid peroxide that is generated as the human body ages.

In particular, the Saururus chinensis according to the present invention is excellent in discharging heavy metals, relieving edema, facilitating diuresis and water management in the body, and in discharging acne (body toxins).

Moreover, flavonoid substances such as quercetin, quercetin, and isoflavones contained in the Saururus chinensis are good for preventing cancer and lifestyle diseases, and quercetin in particular is known to be excellent for detoxification and skin improvement.

As the Saururus chinensis according to the present invention providing the above-described effects, any Saururus chinensis commonly used in the art may be used, but it is recommended to use red clay Saururus chinensis.

Here, the red clay Saururus chinensis is the Endangered Plant No. 177 and is an indigenous plant. It grows very strongly and is a clean plant with no pests or diseases. It is also called Cheon Seong Cho, and its amazing efficacy is recorded in the Ben Cao Gang Mu (Compendium of Materia Medica), Chinese Medicinal Herbs Dictionary, and Donguibogam (Principles and Practice of Eastern Medicine).

Specifically, the Saururus chinensis according to the present invention contains flavonoid substances, water-soluble tannins, essential amino acids, and effective minerals. In particular, the stem contains a large amount of water-soluble tannins, the leaves contain a large amount of quercetin, quercitrin, isoquercetin, avicularin, hyperin, lucin, and water-soluble tannins, and the roots contain various amino acids and effective minerals.

Here, the water-soluble tannins are not only excellent for anticancer, antioxidation, and anti-aging, but also have a diuretic effect and combine with enzymes in the body to burn body fat, while exhibiting anti-inflammatory and anti-tumor effects and detoxification effects. The quercetin, quercitrin, isoquercetin, lucin, and water-soluble tannins are effective in preventing and treating hypertension and stroke, and the flavonoid substances and water-soluble tannins increase the elasticity of peripheral blood vessels, thereby helping peripheral arterial circulation and showing anti-disease and anti-aging effects with diuretic effects.

In particular, the water-soluble tannins have the property of combining well with heavy metals in water along with the above-described effects, so when heavy metals such as mercury or cadmium react with tannin and form tannin mercury and tannin cadmium, the tannins allow the harmful heavy metal components to be excreted from the body without dissolving in the blood, thereby protecting the bodies. In addition, since the water-soluble tannins prevent peroxidation of cells in the body, they strengthens pancreatic cells and has the effect of preventing diabetes.

Green tea and/or puer tea according to the present invention are rich in antioxidant ingredients, such as catechin, which prevent cell damage by removing reactive oxygen species in the body, thereby delaying aging, and are good for preventing chronic diseases such as cancer and stroke. In addition, they lower cholesterol, specifically low-density lipoprotein (LDL) cholesterol levels, and raise high-density lipoprotein (HDL) cholesterol levels, thereby reducing the risk of cardiovascular diseases, and contain caffeine to increase brain activity and improve concentration.

In particular, the green tea according to the present invention contains an amino acid called L-theanine, which helps increase comfort and concentration.

The above-described green tea and/or puer tea are used as materials in the method of preparing aloeswood water according to the present invention to improve the taste of the aloeswood water, in addition to the above-described functions.

The Stachys sieboldii according to the present invention refers to a rhizome of a perennial herbaceous plant belonging to the genus Stachys of the family Lamiaceae. In Korea, the aboveground part of the plant is referred to as seokjampul and the rhizome is referred to as choseokjam.

The Stachys sieboldii is known as a medicinal plant and contains a large amount of choline, which helps prevent dementia and improve memory, as well as phenylethanoids, which help activate brain functions, among various medicinal effects.

The Lycopus lucidus Turcz. according to the present invention refers to the dried whole plant of Lycopus lucidus Turcz., a plant of the family Lamiaceae, which grows widely in moist places in mountains and fields of various regions. The whole plant is mainly used after being cut during the flowering season in summer and dried in the sun, and it is widely used in the oriental medicine to reduce boils or swellings.

The Lycopus lucidus Turcz. contains large amounts of galactooligosaccharides, monoterpenes, sesquiterpenes, and flavonoids, and thus has antioxidant, anti-inflammatory, anti-cancer, analgesic, and sedative effects and is effective in removing edema, removing blood stasis (preventing aggregation of platelets and red blood cells), and enhancing uterine contractility. It also helps ameliorate skin diseases such as itching, atopic dermatitis, and pediatric atopic dermatitis.

The Agrimonia pilosa according to the present invention is a type of mushroom also called straw mushroom, and is distributed in East Asia including Korea, China, and Japan, and has been widely used as a medicinal mushroom since long ago.

The Agrimonia pilosa contains large amounts of catechin, which has antioxidant and anticancer effects, and water-soluble tannins such as agrimonin and/or agrimonolide, which have anti-inflammation, anticancer, and antibacterial effects and help hemostasis and diarrhea.

Furthermore, the Agrimonia pilosa is known to improve skin elasticity, reduce wrinkles, and have a whitening effect in addition to the above-described effects.

The Panax notoginsengs according to the present invention refers to the root of Panax notoginsengs, a perennial herbaceous plant belonging to the family Araliaceae. It is mainly distributed in Yunnan and Guangxi provinces of China, and is known as an important medicinal herb with a long history.

The Panax notoginsengs generally has a long and cylindrical shape, brown or red in color, and has fine hairs curved stripes on its surface

The Panax notoginsengs contains various medicinal ingredients such as triterpenoid saponins, ginseng saponins, polyphenols, flavonoids, amino acids, vitamins, and minerals, and exhibits effects such as hemostasis, anti-inflammation, antithrombosis, anti-arteriosclerosis, hematopoiesis promotion, and/or liver protection.

The method of preparing aloeswood water according to the present invention having the above-described components, specifically, the method of preparing aloeswood water containing aloeswood, and more specifically, the method of preparing aloeswood water containing aloeswood with atopic dermatitis and acne ameliorating effects include: a material preparation step in which materials are prepared; a material pulverization step in which materials are pulverized; a boiling step in which the pulverized materials are mixed at a specific ratio and boiled with water; an infusing step in which the mixture boiled through the boiling step is infused for a certain period of time; and a reduced pressure distillation step in which the infused water is distilled under reduced pressure.

The material preparation step according to the present invention includes washing the materials used, specifically, aloeswood, Phellinus linteus, Houttuynia cordata Thunb., Saururus chinensis, green tea or puer tea, Stachys sieboldii, Lycopus luci, Agrimonia pilosa, and Panax notoginsengs, and then drying them in the shade, but preferably, washing them in flowing water and then drying them in the shade for 45 to 50 hours, preferably about 48 hours.

The material pulverization step according to the present invention includes pulverizing each of the aloeswood, Phellinus linteus, Houttuynia cordata Thunb., Saururus chinensis, green tea or puer tea, Stachys sieboldii, Lycopus luci, Agrimonia pilosa, and Panax notoginsengs, dried through the material preparation step.

Here, for the pulverization in the material pulverization step, any pulverization method commonly used in the art may be used, but it is recommended to include pulverizing each of the dried materials using a ball mill.

The boiling step according to the present invention includes, after the material pulverization step is completed, mixing aloeswood powder, mulberry Phellinus linteuspowder, Houttuynia cordata Thunb. powder, Saururus chinensis powder, green tea powder or puer tea powder, Stachys sieboldii powder, Lycopus lucidus Turcz. powder, Agrimonia pilosa powder, and Panax notoginsengs powder are mixed at a weight ratio of 3:1.3 to 1.7:1.3 to 1.7:1.1 to 1.5:0.8 to 1.2:0.3 to 0.7:0.5 to 0.9:0.1 to 0.5:0.1 to 0.4, as recommended, at a weight ratio of 3:1.5:1.5:1.3:1:0.5:0.7:0.3:0.2, to prepare a material mixture, mixing water in an amount of 9,000 to 11,000 parts by weight based on 100 part by weight of the material mixture, and heating and boiling the resulting mixture in a temperature range of 80 to 100° C. for 22 to 26 hours.

Here, the water may be purified water containing minerals that help alcohol decomposition or human metabolism, for example, purified water containing mineral ions of zinc, copper, silver, iron, and tin.

At this time, the ionization of the minerals is achieved by high-voltage electrolysis, and the mineral ions may be contained in purified water preferably at 300 ppm or less.

The infusing step according to the present invention includes, after the boiling step is completed, performing infusion by keeping the boiled water in a temperature range of 15 to 25° C., preferably at a temperature of about 25° C., for 22 to 26 hours, preferably for about 24 hours.

The reduced pressure distillation step according to the present invention includes, after the boiling step is completed, distilling the infused water under reduced pressure.

Here, the reduced pressure distillation is performed to produce aloeswood water by removing contaminants such as heavy metals that may be contained in the water by any chance, by distilling the water using a reduced pressure distillation method.

At this time, as the reduced pressure distillation method, any conventional reduced pressure distillation methods in the art may be used, but the method preferably includes reduced pressure distillation at a pressure of the distillation vessel in the range of 0 to 760 mmHg.

When necessary, the reduced pressure distillation may include reduced pressure distillation at a temperature range of 80 to 100° C.

In a specific embodiment, the reduced pressure distillation may include using an automatic mechanical device facility (distillation facility).

The method of preparing aloeswood water according to the present invention, specifically, the method of preparing aloeswood water containing aloeswood, and more specifically, the method of preparing aloeswood water containing aloeswood with atopic dermatitis and acne ameliorating effects may further include one or more additives according to the specific embodiment described below.

At this time, it is preferable to use the medicinal herbs among the additives after washing and drying them in the same manner as in the material preparation step of the present invention.

In a specific embodiment, the method of preparing aloeswood water containing aloeswood, and more specifically, the method of preparing aloeswood water containing aloeswood with atopic dermatitis and acne ameliorating effects may further include 1 to 10 parts by weight of ballon flower dietary fiber based on 100 parts by weight of the material mixture in the boiling step.

The balloon flower has been widely used for a long time not only as food but also as medicine. It first appeared in the Hyangyak Jipseongbang (Compendium of Korean Prescriptions) among the literary records. It has a spicy and mild taste and is slightly toxic. It is said that the roots are dug up in February to August, and the roots dried in the sun are good for sore throat. Donguibogam (Principles and Practice of Eastern Medicine) says that it has a slightly cold nature, a spicy and bitter taste, and is slightly toxic, and it treats diseases of the lungs, throat, nose, and chest and removes insect venom. When the ballon flower dietary fiber is used, in addition to the above-described effects of the balloon flower, the effects of dietary fiber intake, such as helping the movement of the large intestine, increasing the amount of defecation, and preventing lifestyle diseases such as diabetes and hypertension, may be added. Therefore, the balloon flower dietary fiber may be extracted and used as described below.

The extraction of the dietary fiber of the balloon flower according to the present invention may be obtained through the following process. First, the raw balloon flower is wet-heat sterilized at a steam temperature of 90 to 100° C. for 1 to 10 minutes and then dried to a moisture content of 5% to 25%, and the dried balloon flower is pulverized into powder by a ball mill, water is added to extract the finely pulverized balloon flower, and then the balloon flower dietary fiber may be extracted using a centrifuge.

Here, the purpose of wet-heat sterilizing the balloon flower and then drying it is to facilitate the removal of impurities and pulverization, and the purpose of using a centrifuge is to effectively extract the balloon flower dietary fiber from the finely pulverized balloon flower.

In another specific embodiment, the method of preparing aloeswood water containing aloeswood according to the present invention may further include 3 to 7 parts by weight of fermented Bidens tripartita based on 100 parts by weight of the material mixture in the boiling step.

The Bidens tripartita is a perennial herb widely distributed in East Asia including Korea, China, and Japan, and it grows in humid mountains and valleys and blooms from June to August, and the fruit ripens in September. Its main efficacies include lowering blood pressure, improving vascular health, reducing cholesterol, exhibiting an anti-inflammatory effect, and improving skin health. The preparation process is described below.

First, dried Bidens tripartita is crushed into pieces in a size of 0.5 to 5 mm, then the crushed Bidens tripartita is mixed with sugar at a weight ratio of 50:40.

At this time, the purpose of crushing Bidens tripartita pieces in a size of 0.5 to 5 mm is to ensure that the sugar is evenly mixed into the Bidens tripartita, and the weight ratio of the Bidens tripartita and sugar is 50:40 to prevent the sugar from being mixed excessively and making the sweetness too strong, and to prevent the solid matter of the Bidens tripartita from being too soft.

Next, the sugar mixed with Bidens tripartite is allowed to stand so that the sugar may be dissolved and sufficiently infiltrated, and the resulting mixture is transferred into an earthenware jar.

Next, the mouth to the earthenware jar is covered with a cloth and sealed, and the mixture is fermented for about six months and then the solid matter is filtered out.

In another specific embodiment, the method of preparing aloeswood water containing aloeswood according to the present invention may further include 3 to 7 parts by weight of a red bean extract based on 100 parts by weight of the material mixture in the boiling step.

The red bean is a dicotyledonous plant belonging to the family Leguminosas of the order Rosales. Red bean is rich in vitamin B1, so when mixed with rice, it provides vitamins that are often lacking in rice. It is effective in preventing beriberi as well as recovering from fatigue. Most of its protein is glycine, and it is rich in essential amino acids except for valine. In particular, it is known to have a high content of lysine, which is an amino acid limited in rice, so when mixed with rice, it improves the quality of protein due to the amino acid supplement effect. In addition, in the oriental medicine, red bean is plain in nature and sweet in taste, and has the effects of clearing heat and detoxifying toxins, so it is used for edema, beriberi, urinary incontinence, and sores and toxins. The saponins contained in red bean, together with fiber, have the effect of aiding bowel movements, thereby removing toxins, promoting bowel movements, and cleansing the intestines, and are also used for kidney disease, beriberi, hangovers, etc. The pigment of red beans is known to be cyanidin, which is a type of anthocyanin, and the antioxidant and antitumor effects of the pigment have already been experimentally proven. In particular, polyphenol components such as proanthocyanidins and quercetin glycosides, which are abundant in red beans, have been reported to exhibit strong antioxidant and anti-inflammatory effects. The preparation process of a red bean extract is described below.

First, red beans are washed and roasted at a temperature of 150 to 200° C. for 30 minutes, cooled at room temperature for 1.5 to 3 hours, pulverized with a ball mill to prepare powder, and the powdered red beans are placed in an extractor and extracted at a temperature of 120 to 180° C. and a pressure of 3 to 5 kgf/cm² for two to four hours to prepare an extract.

Here, the purpose of washing the red beans is to remove foreign substances, the purpose of roasting the red beans at a temperature of 150 to 200° C. for 30 minutes is to remove the astringent taste of raw red beans and make them easier to digest when consumed, the purpose of pulverization is to evenly mix the effective ingredients of the red bean skin and kernel, and the purpose of extracting in an extractor at a temperature of 120 to 180° C. and a pressure of 3 to 5 kgf/cm² for two to four hours is to extract the effective ingredients of the red beans as effectively as possible.

In another specific embodiment, the method of preparing aloeswood water containing aloeswood according to the present invention may further include 1 to 10 parts by weight of a fermented garlic extract based on 100 parts by weight of the material mixture in the boiling step.

In the fermented garlic, the content of polyphenols increases, and as the extraction becomes easier, the content of water-soluble sulfur-containing amino acids such as S-allyl-cysteine (SAC) increases, so that the antioxidant function increases compared to raw garlic, and it can provide effects such as cancer prevention, cholesterol reduction, arteriosclerosis amelioration, heart disease prevention, oxidative stress-associated effects, and anti-inflammatory activity.

At this time, any garlic that is common in the art may be used, but it is recommended to use whole garlic with its skin intact to increase the content of SAC, which may increase its efficacy. The preparation process is described below.

First, 500 to 1,000 parts by weight of water is added to 100 parts by weight of garlic, and the garlic is soaked in hot water. Then, the garlic soaked in hot water is cooled to room temperature, and 1 to 5 parts by weight of Lactobacillus plantarum lactic acid bacterium is added based on 100 parts by weight of garlic to ferment for one to three days. The product is filtered, and then placed in an extractor and extracted at a temperature of 120 to 180° C. and under a pressure of 3 to 5 kgf/cm² for three hours to obtain a first extract. Then, the first extract is filtered, and the residue excluding the first extract is used to obtain a second extract in the same manner as the first extraction, and then the first extract and the second extract are mixed to prepare a mixture.

Here, 500 to 1,000 parts by weight of water is added based on 100 parts by weight of garlic, and the garlic is soaked in hot water because, when less than 500 parts by weight of water is added based on 100 parts by weight of garlic, the yield is low, which may cause a problem in that the effect of adding the garlic extract is minimal, and when more than 1,000 parts by weight is added, the yield is high, but the content of active ingredients such as SAC may decrease, which is not good.

In addition, lactic acid bacterium is added in an amount of 1 to 5 parts by weight based on 100 parts by weight of garlic and fermenting is performed for one to three days, because when the amount of lactic acid bacterium is less than 1 part by weight based on 100 parts by weight of garlic, the amount of enzyme secreted from the added lactic acid bacterium may be too little, making it difficult to smoothly separate and convert SAC present in the hot water-soaked garlic product, and when the amount is more than 5 parts by weight, other substances other than SAC may be extracted together at a high content, and so the production cost may be increased, which is not good. The purpose of dividing the fermented garlic into first and second extractions is to maximize the utilization of the active ingredients by extracting once again the active ingredients of the garlic remaining after the first extraction through the second extraction.

In another specific embodiment, the method of preparing aloeswood water containing aloeswood according to the present invention may further include 1 to 10 parts by weight of a peanut shell extract based on 100 parts by weight of the material mixture in the boiling step.

The peanut is a dicotyledonous plant native to the region and belonging to the family Leguminosae of the order Rosales. The peanut shell is rich in unsaturated fatty acids, proteins, dietary fiber, vitamin E, and antioxidants, and is known to help improve cardiovascular health, control blood sugar, improve digestive health, manage weight, prevent cancer, improve brain health, and improve skin health. The preparation process is described below.

First, 800 to 1,000 parts by weight of water is added to 100 parts by weight of peanut shells, and the resulting mixture is placed in a reflux extractor, refluxed at 81 to 83° C. for three hours under a pressure of 3 to 4 kgf/cm², filtered through a filter net, and then filtered again to produce the product.

Here, the purpose of using 800 to 1,000 parts by weight of water based on 100 parts by weight of peanut shells is to effectively swell the peanut shells and discharge the active ingredients contained in the peanut shells, and the purpose of placing the peanut shells in a reflux extractor and performing reflux extraction at 81 to 83° C. for three hours under a pressure of 3 to 4 kgf/cm², then filtering through a filter net, and then filtering again is to effectively extract the active ingredients while preventing the active ingredients of the peanut shells from being destroyed by heat, and also to remove foreign substances attached to the extract.

In another specific embodiment, the method of preparing aloeswood water containing aloeswood according to the present invention may further include 8 to 12 parts by weight of Huperzia serrata based on 100 parts by weight of the material mixture in the boiling step.

Here, the Huperzia serrata is a medicinal herb with a long history, and has been introduced in various medical fields, including Chinese medicine, and has anti-acetylcholinesterase properties.

At this time, the anti-acetylcholinesterase has been actively used in countries around the world since the 1980s for its efficacy in treating and preventing dementia, and in China, it is mainly used for Alzheimer's disease, and recently, it has been shown to beneficially improve cognitive functions.

Moreover, the Huperzia serrata has recently been sought out by the a French medicinal herb research institute in Vietnam, the native region thereof, to obtain this herb as an effective agent for preventing and treating dementia.

In another specific embodiment, the method of preparing aloeswood water containing aloeswood according to the present invention may further include 6 to 10 parts by weight of vegetable worms based on 100 parts by weight of the material mixture in the boiling step.

Here, the vegetable worm is a health supplementary food mainly produced in the Chinese highlands of Tibet, at altitudes of about 4,000 m or higher, and is rich in protein, vitamins, calcium, iron, potassium, sodium, and the like. Its efficacy is to control blood pressure and blood sugar, and it also has antibacterial and anticancer effects. It is also widely known as an excellent diet food because it is a very low-calorie food in terms of nutrition.

In another specific embodiment, the method of preparing aloeswood water containing aloeswood according to the present invention may further include 3 to 7 parts by weight of lecithin based on 100 parts by weight of the material mixture in the boiling step.

The lecithin has a highly lipophilic fatty acid group on one side and a highly hydrophilic choline moiety on the other side, and thus it may stabilize water and a mixture.

In particular, the lecithin is a major component that constitutes biological membranes. It increases the activity of cell membranes to slow down cell destruction, stimulates tissue activity to prevent decline in brain activity, and helps the absorption of fat-soluble vitamins (A, D, K) as well as vitamin E to increase their efficiency in the body, thereby preventing skin aging and promotes smooth blood circulation. In addition, it is also useful for health as it is involved in the solubilization of cholesterol and reduces the amount of blood cholesterol level, thereby being effective in preventing cardiovascular diseases such as myocardial infarction.

In another specific embodiment, the method of preparing aloeswood water containing aloeswood according to the present invention may further include 8 to 12 parts by weight of Liriope platyphylla powder based on 100 parts by weight of the material mixture in the boiling step.

Here, the Liriope platyphylla is a perennial herbaceous plant belonging to the family Liliaceae that grows well in shady and moist places, blooms purple flowers in July, and bears black fruits in October. It has been reported that the efficacy of Liriope platyphylla includes hypoglycemic, anti-inflammatory, anti-arrhythmic, or anticancer effects. In addition, it has been reported that the main active ingredients of Liriope platyphylla include steroidal saponins such as spicatoside A and B, ophiopogenine A and B, methylophiopogonone A and B, ophiopogonanone A, and homo-isoflavonoids such as methylophiopoginanone A and B, and that β-sitosterol, stigmasterol, β-sitosterol glucoside, and various polysaccharides such as oligosaccharides are contained in 60% or more of the tuberous root. The preparation process of the Liriope platyphylla powder is described below.

First, Liriope platyphylla is washed, frozen, then thawed gently. Then, the thawed Liriope platyphylla is steamed in a steamer for 15 to 25 minutes, placed in a heat insulating container and kept warm for 1.5 to 2.5 days to perform pre-ripening. Then, the pre-ripened Liriope platyphylla is taken out and dried in a well-ventilated tray for 24 to 36 hours. Then, the dried Liriope platyphylla is placed in a heat insulating container and kept warm for four to six days to perform post-ripening. It is recommended to use it after pulverizing into powder.

Here, the pre-ripening process of Liriope platyphylla facilitates the extraction of the active ingredients of Liriope platyphylla, resolves side effects such as indigestion and nausea that occur when consuming Liriope platyphylla, and facilitates pulverization into powder, while ensuring that the active ingredients are evenly distributed through the post-ripening process.

In another specific embodiment, the method of preparing aloeswood water containing aloeswood according to the present invention may further include 8 to 12 parts by weight of apple powder based on 100 parts by weight of the material mixture in the boiling step.

The apple powder contains fructose, glucose, organic acids, and the like, so it may be consumed by the human body to improve health.

Here, any apple powder commonly used in the art may be used as the apple powder, but it is recommended to use apples cut into appropriate sizes with the peel thereon, freeze-dried, and powdered.

At this time, the purpose of freeze-drying the apple with the peel thereon is to preserve the fructose, organic acid, and glucose contained in the apple without being destroyed or damaged, thereby increasing its efficacy.

Meanwhile, in addition to freeze-drying the apples, they may also be quickly dried in a dryer or dried at room temperature directly in the sun or indirectly indoors.

In another specific embodiment, the method of preparing aloeswood water containing aloeswood according to the present invention may further include 1 to 10 parts by weight of Forsythiae fructus based on 100 parts by weight of the material mixture in the boiling step.

The Forsythiae fructus refers to the fruit of the forsythia, a plant in the family Oleaceae, and it is usually picked when the fruit is first ripe or fully ripe and dried in the sun before use.

In particular, the Forsythiae fructus is mainly used in oriental medicine as an antipyretic, detoxifying, draining, anti-inflammatory, and diuretic agent, and is used together with other medicinal herbs for purulent diseases, swelling, gonorrhea, menstruation, diuresis, hemorrhoids, tuberculosis, scabies, and detoxification. The extract or decomposition product of the fruit peel is known to have antibacterial effects, and the flower contains the pigment glycosides quercetin, glucoside, rutin, and ascorbic acid, which are known to have antioxidant, antibacterial, anti-inflammatory, and elastin-decomposing enzyme inhibiting effects, which are good for women's beauty.

Therefore, the Forsythiae fructus extract according to the present invention may provide an excellent whitening effect due to its high effect in tyrosinase activity inhibition and melamine production.

In another specific embodiment, the method of preparing aloeswood water containing aloeswood according to the present invention may further include 3 to 7 parts by weight of Styphnolobium japonicum powder based on 100 parts by weight of the material mixture in the boiling step.

The Styphnolobium japonicum is a deciduous broad-leaved tree belonging to the family Leguminosae, and is rich in sophoricoside, a type of isoflavone, so it has excellent physiological activity and may help improve menopausal symptoms and provide other health benefits.

In another specific embodiment, the method of preparing aloeswood water containing aloeswood according to the present invention may further include 3 to 5 parts by weight of Vitex agnus-castus based on 100 parts by weight of the material mixture in the boiling step.

The Vitex agnus-castus may be used in the form of bark, roots, leaves, and fruits, and because it contains flavonoids and iridoids, it may help with overall adult health, such as improving menopausal health by normalizing hormonal function, ameliorating female menstrual irregularities, and improving internal secretion, In particular, it is effective in treating physical and mental symptoms such as headaches, skin symptoms, breast swelling, lower abdominal pain, hypernoia, oversensitive emotional fluctuations, irritability, depression, fatigue, and sleep disorders caused by premenstrual syndrome (PMS), and ameliorating menopausal symptoms such as ameliorating menstrual irregularities.

In another specific embodiment, the method of preparing aloeswood water containing aloeswood according to the present invention may further include 3 to 7 parts by weight of cassia seed powder based on 100 parts by weight of the material mixture in the boiling step.

The cassia seed is known to be sweet and bitter in taste and cold in nature, and is well known to act on the liver, large intestine, and kidney meridians to reduce liver heat and brighten the eyes. It is effective for people with bloodshot eyes and calcaneodynia due to liver fire or wind heat, and for those with poor eyesight or acute conjunctivitis.

In particular, the cassia seeds have the effect of lubricating the intestines to promote bowel movements and regulating the liver's energy to calm energy, and have a cholesterol-lowering effect, and thus are helpful for patients with hyperlipidemia or hypertension.

In another specific embodiment, the method of preparing aloeswood water containing aloeswood according to the present invention may further include 3 to 7 parts by weight of fermented ginseng based on 100 parts by weight of the material mixture in the boiling step.

Here, the ginseng is a medicinal plant belonging to the family Araliaceae, and is known to provide effects such as increasing immunity, relieving fatigue, improving memory, improving blood circulation through inhibition of platelet aggregation, and antioxidant effects. Fermented ginseng may have similar medicinal effects to red ginseng made from ginseng, and the preparation method is described below.

First, ginseng is washed and dehydrated, then cut into small pieces, steamed in a steamer at about 121° C. for about 20 minutes, cooled to about 35 to 40° C., and then evenly mixed and fermented with 0.5 to 2 parts by weight of fermentation bacteria, for example, one or more microorganisms selected from the group consisting of Saccharomyces cerevisiae, Bacillus amyloliquefaciens, and Lactobacillus plantarum, based on 100 parts by weight of ginseng, and then the resulting product is naturally dried.

At this time, the naturally dried product may be further aged by wrapping it in Korean traditional paper or similar and allowing it to stand at room temperature for 12 to 36 hours.

Here, the purpose of washing, dehydrating, and cutting the ginseng is to remove foreign substances such as dirt attached to the ginseng, the purpose of steaming it in a steamer is to remove bacteria, and the purpose of inoculating it with a microorganism and drying it is to improve the fermentation efficiency.

In another specific embodiment, the method of preparing aloeswood water containing aloeswood according to the present invention may further include 3 to 7 parts by weight of Humulus japonicus extract based on 100 parts by weight of the material mixture in the boiling step.

Here, the Humulus japonicus is a perennial vine native to East Asian regions including Korea, Japan, and China, and has been used as a medicinal plant for a long time, and has recently become popular as a health functional food.

In particular, the main components of the Humulus japonicus include ginsenosides, flavonoids, polysaccharides, and the like, and the ginsenoside is a representative active ingredient of Humulus japonicus and is known to have effects such as antioxidant, anti-inflammatory, and immunity-enhancing effects. The flavonoid is known to have an excellent antioxidant effect to help prevent cell damage and aging, and the polysaccharide is known to help enhance immunity and control blood sugar.

A preferred Humulus japonicus extract is not particularly limited as long as it is a Humulus japonicus extract prepared by a conventional method in the art, but preferably, the Humulus japonicus extract is prepared by placing Humulus japonicus in an extractor and extracting under a pressure of 3 to 4 kgf/cm² and at a temperature of 60 to 80° C. for one to two hours, then placing the extract in a stirrer and stirring it for 10 to 12 hours to remove moisture so that the active ingredients may remain in an amount of 60% by weight (water content of about 40%) based on 100% by weight of the total Humulus japonicus extract, thereby obtaining a concentrated extract.

Here, the purpose of placing Humulus japonicus into an extractor and extracting under a pressure of 3 to 4 kgf/cm² and at a temperature of 60 to 80° C. for one to two hours is to prevent the active ingredients of the Humulus japonicus from being destroyed when the extraction temperature is too high, and the purpose of retaining the active ingredients in an amount of 60% by weight based on 100% by weight of the total Humulus japonicus extract is to efficiently concentrate the active ingredients of the Humulus japonicus.

In another specific embodiment, the method of preparing aloeswood water containing aloeswood according to the present invention may further include 3 to 7 parts by weight of Paeoniae radix Alba powder based on 100 parts by weight of the material mixture in the boiling step.

Here, the Paeoniae radix Alba is a root of the plant of the family Paeoniaceae, which is boiled and dried after removing the pericarp, and may be prepared into Paeoniae radix Alba powder by pulverizing it.

The aloeswood water prepared in this manner is a solution in which the active ingredients of aloeswood are stably dispersed, and may be used for various purposes, including as a beverage ingredient.

When a beverage is prepared using the aloeswood water according to the present invention, a beverage that is easy to drink without discomfort may be made by adding other nutritional ingredients or adding a sweetener commonly used in beverages to the aloeswood water.

At this time, the types of the added nutritional ingredients and/or sweeteners are not particularly limited, but it is recommended that vitamins, citric acid, phosphoric acid, lactic acid bacteria, glucitol, saccharin, sucralose, xylitol, aspartame, magnesium silicate, calcium silicate, crystalline cellulose, powdered cellulose, silicon dioxide, or at least one or more mixtures selected from these be further included, and the amount used may be changed according to the user's choice.

Hereinafter, the present invention will be specifically described through examples. However, the following examples are only intended to specifically describe the present invention, and the scope of the present invention is not limited by these examples.

### EXAMPLE 1

Aloeswood, mulberry Phellinus linteus, Houttuynia cordata Thunb., Saururus chinensis, green tea, Stachys sieboldii, Lycopus luci, Agrimonia pilosa, and Panax notoginsengs were washed with water and then dried in the shade for about 48 hours to dry the materials.

Next, the dried aloeswood, mulberry Phellinus linteus, Houttuynia cordata Thunb., Saururus chinensis, green tea, Stachys sieboldii, Lycopus luci, Agrimonia pilosa, and Panax notoginsengs were pulverized into powder with a ball mill.

Next, 100 g of material mixture was prepared by mixing 30 g of the pulverized aloeswood powder, 15 g of mulberry Phellinus linteus powder, 15 g of Houttuynia cordata Thunb. powder, 13 g of Saururus chinensis powder, 10 g of green tea, 5 g of Stachys sieboldii powder, 7 g of Lycopus luci powder, 3 g of Agrimonia pilosa powder, and 2 g of Panax notoginsengs powder, and the mixture was mixed with 10,000 g (10 L) of water, and the resulting mixture was boiled at a temperature of about 90° C. for about 24 hours.

Next, the boiled water was infused at a temperature of about 20° C. for about 24 hours.

Next, the infused water was distilled under reduced pressure at a pressure of 400 mmHg to prepare aloeswood water.

### [Preparation of Balloon Flower Dietary Fiber]

100 g of balloon flower was selected and wet-heat sterilized with at a steam temperature of 95° C. for five minutes, then dried to a moisture content of 15%.

Next, the dried balloon flower was ground into powder using a ball mill.

Next, 1,000 g of water was added to the powdered balloon flower to make the balloon flower fine.

Then, the fined balloon flower was extracted using a centrifugal filter to prepare balloon flower dietary fiber.

### EXAMPLE 2

Example 2 was implemented in the same manner as Example 1, except that 5 g of the balloon flower dietary fiber prepared according to the above [Preparation of balloon flower dietary fiber] was further added to 100 g of the material mixture.

[Preparation of Fermented Bidens tripartita]

Bidens tripartita was pulverized into a size of about 3 mm.

Next, the pulverized Bidens tripartita was mixed with sugar at a weight ratio of 50:40.

Next, the sugar mixed with Bidens tripartite was allowed to stand so that the sugar may be dissolved and sufficiently infiltrated, and the resulting mixture was transferred into an earthenware jar.

Next, the mouth to the earthenware jar was covered with a cloth and sealed, and the mixture was fermented for about six months.

Next, the solid matter was filtered out to prepare fermented Bidens tripartita.

### EXAMPLE 3

Example 3 was implemented in the same manner as Example 1, except that 5 g of the fermented Bidens tripartita prepared according to the above [Preparation of fermented Bidens tripartita] was further added to 100 g of the material mixture.

### [Preparation of Red Bean Extract]

100 g of red beans were washed, roasted at 180° C. for 30 minutes, cooled at room temperature for two hours, pulverized with a ball mill to make powder, and the powdered red beans were placed in an extractor and extracted at 150° C. and under a pressure of 4 kgf/cm² for three hours to prepare a red bean extract.

### EXAMPLE 4

Example 4 was implemented in the same manner as Example 1, except that 5 g of the red bean extract prepared according to the above [Preparation of red bean extract] was further added to 100 g of the material mixture.

### [Preparation of Fermented Garlic Extract]

700 g of water was added to 100 g of garlic, and the garlic was soaked in hot water. Then, the garlic soaked in hot water was cooled to room temperature, and 3 g of Lactobacillus plantarum lactic acid bacterium was added to ferment for two days. The product was filtered, and then placed in an extractor and extracted at a temperature of 150° C. and under a pressure of 4 kgf/cm² for three hours to obtain a first extract. Then, the first extract was filtered, and 700 g of water was added to the residue obtained after the filtering. The resulting mixture was soaked in hot water and then cooled to room temperature. 3 g of Lactobacillus plantarum lactic acid bacterium was added to ferment for two days. Then, The product was filtered, and then placed in an extractor and extracted at a temperature of 150° C. and under a pressure of 4 kgf/cm² for three hours to obtain a second extract. The first extract and the second extract are then mixed to prepare a fermented garlic extract.

### EXAMPLE 5

Example 5 was implemented in the same manner as Example 1, except that 5 g of

the fermented garlic extract prepared according to the above [Preparation of fermented garlic extract] was further added to 100 g of the material mixture.

### [Preparation of Peanut Shell Extract]

900 g of water was added to 100 g of peanut shells, and the resulting mixture was placed in a reflux extractor and refluxed at 82° C. for three hours under a pressure of 3.5 kgf/cm². Then, the product was filtered through a filter net, and then filtered again through a filter net to prepare a peanut shell extract.

### EXAMPLE 6

Example 6 was implemented in the same manner as Example 1, except that 5 g of the fermented garlic extract prepared according to the above [Preparation of fermented garlic extract] was further added to 100 g of the material mixture.

### EXAMPLE 7

Example 7 was implemented in the same manner as Example 1, except that 10 g of Huperzia serrata powder was further added to 100 g of the material mixture.

### EXAMPLE 8

Example 8 was implemented in the same manner as Example 1, except that 8 g of vegetable worm powder was further added to 100 g of the material mixture.

### EXAMPLE 9

Example 9 was implemented in the same manner as Example 1, except that 5 g of lecithin was further added to 100 g of the material mixture.

[Preparation of Liriope platyphylla Powder]

Liriope platyphylla was washed, frozen, then thawed gently.

Next, the thawed Liriope platyphylla was steamed in a steamer for 20 minutes, placed in a heat insulating container and kept warm for two days to perform pre-ripening.

Next, the pre-ripened Liriope platyphylla was dried in tray for 24 hours, and then kept warm for five days for ripening. Then, it was pulverized into powder using a ball mill.

### EXAMPLE 10

Example 10 was implemented in the same manner as Example 1, except that 10 g of the Liriope platyphylla powder prepared was further added to 100 g of the material mixture.

### EXAMPLE 11

Example 11 was implemented in the same manner as Example 1, except that 10 g of apple powder prepared by cutting apples with the peel thereon and freeze-drying and then pulverizing the cut apples was further added to 100 g of the material mixture.

### EXAMPLE 12

Example 12 was implemented in the same manner as Example 1, except that 5 g of Forsythia fructus extract was further added to 100 g of the material mixture.

### EXAMPLE 13

Example 13 was implemented in the same manner as Example 1, except that 5 g of Styphnolobium japonicum powder was further added to 100 g of the material mixture.

### EXAMPLE 14

Example 14 was implemented in the same manner as Example 1, except that 5 g of Vitex agnus-castus powder was further added to 100 g of the material mixture.

### EXAMPLE 15

Example 15 was implemented in the same manner as Example 1, except that 5 g of cassia seed powder was further added to 100 g of the material mixture.

### [Preparation of Fermented Ginseng]

100 g of ginseng was washed and dehydrated, then cut into small pieces.

Next, the cut ginseng was steamed in a steamer at 121° C. for 20 minutes, cooled to about 38° C., and then 1 g of Saccharomyces cerevisiae was inoculated, evenly mixed, and fermented.

Next, the fermented ginseng was naturally dried, and further aged for 24 hours by wrapping it in Korean traditional paper.

### EXAMPLE 16

Example 16 was implemented in the same manner as Example 1, except that 5 g of the ginseng extract prepared according to the above [Preparation of fermented ginseng] was further added to 100 g of the material mixture.

[Preparation of Humulus japonicus Extract]

Humulus japonicus was cleanly washed.

Next, the Humulus japonicus was placed in an extractor and extracted under a pressure of 3.5 kgf/cm² and at a temperature of 65° C. for 1.5 hours to prepare Humulus japonicus extract.

Next, the Humulus japonicus extract was placed in a stirrer and stirred for 11 hours to remove moisture so that the active ingredients remained in an amount of 60% by weight based on 100% by weight of the total Humulus japonicus extract, thereby obtaining a concentrated extract.

### EXAMPLE 17

Example 17 was implemented in the same manner as Example 1, except that 5 g of the Humulus japonicus extract prepared according to the above [Preparation of Humulus japonicus extract] was further added to 100 g of the material mixture.

### EXAMPLE 18

Example 18 was implemented in the same manner as Example 1, except that 5 g of Paeoniae radix Alba powder was further added to 100 g of the material mixture.

### EXAMPLE 19

Example 19 was implemented in the same manner as Example 1, except that all the additives according to Examples 2 to 18 were added.

### EXAMPLE 20

Example 20 was implemented in the same manner as Example 1, except that 10,000 g (10 L) of purified water containing minerals was used instead of 10,000 g (10 L) of water.

### EXPERIMENT 1

For each of the aloeswood water products prepared according to the examples, an experiment was conducted with 21 experimental participants with chronic atopic dermatitis or acne for a total of 3 months.

Of the 21 participants for each of the aloeswood water products, 7 were children less than 13 years of age, 7 were adolescents aged 13 to 18 years, and the remaining 7 were adults aged 18 years or older.

These experimental participants had atopic dermatitis symptoms that were particularly severe during the dry season from fall to winter and spring, and the experimental results of the present invention obtained through these participants are as shown in Table 1 below. These experimental participants had suffered from atopic 10 dermatitis or acne for a long time, and therefore no separate control group was used.

TABLE 1

| | | | Degree of | |
|---|---|---|---|---|
| | | | satisfaction | |
| | | | (ratio of | |
| | | | satisfied | Detection of |
| | Symptoms | Ameliorated symptoms | participants) | heavy metals |
| Example 1 | atopic dermatitis | atopic dermatitis and | 16/21 | not detected |
| | and acne | acne ameliorated | | |
| Example 2 | atopic dermatitis | atopic dermatitis and | 15/21 | not detected |
| | and acne | acne ameliorated | | |
| Example 3 | atopic dermatitis | atopic dermatitis and | 15/21 | not detected |
| | and acne | acne ameliorated | | |
| Example 4 | atopic dermatitis | atopic dermatitis and | 16/21 | not detected |
| | and acne | acne ameliorated | | |
| Example 5 | atopic dermatitis | atopic dermatitis and | 16/21 | not detected |
| | and acne | acne ameliorated | | |
| Example 6 | atopic dermatitis | atopic dermatitis and | 15/21 | not detected |
| | and acne | acne ameliorated | | |
| Example 7 | atopic dermatitis | atopic dermatitis and | 17/21 | not detected |
| | and acne | acne ameliorated | | |
| Example 8 | atopic dermatitis | atopic dermatitis and | 15/21 | not detected |
| | and acne | acne ameliorated | | |
| Example 9 | atopic dermatitis | atopic dermatitis and | 15/21 | not detected |
| | and acne | acne ameliorated | | |
| Example 10 | atopic dermatitis | atopic dermatitis and | 17/21 | not detected |
| | and acne | acne ameliorated | | |
| Example 11 | atopic dermatitis | atopic dermatitis and | 16/21 | not detected |
| | and acne | acne ameliorated | | |
| Example 12 | atopic dermatitis | atopic dermatitis and | 15/21 | not detected |
| | and acne | acne ameliorated | | |
| Example 13 | atopic dermatitis | atopic dermatitis and | 18/21 | not detected |
| | and acne | acne ameliorated | | |
| Example 14 | atopic dermatitis | atopic dermatitis and | 17/21 | not detected |
| | and acne | acne ameliorated | | |
| Example 15 | atopic dermatitis | atopic dermatitis and | 15/21 | not detected |
| | and acne | acne ameliorated | | |
| Example 16 | atopic dermatitis | atopic dermatitis and | 16/21 | not detected |
| | and acne | acne ameliorated | | |
| Example 17 | atopic dermatitis | atopic dermatitis and | 17/21 | not detected |
| | and acne | acne ameliorated | | |
| Example 18 | atopic dermatitis | atopic dermatitis and | 15/21 | not detected |
| | and acne | acne ameliorated | | |
| Example 19 | atopic dermatitis | atopic dermatitis and | 16/21 | not detected |
| | and acne | acne ameliorated | | |
| Example 20 | atopic dermatitis | atopic dermatitis and | 13/21 | not detected |
| | and acne | acne ameliorated | | |

As shown in Table 1, in the examples where the method of preparing aloeswood water containing aloeswood was used, no heavy metals were detected, atopic dermatitis and acne ameliorating effects were found, and the degree of satisfaction was high.

### EXPERIMENT 2

Fifty men and women in their 20 s, 30 s, and 40 s were selected, and 30 people in total were selected, and 10 people were allocated to each age group, which included participants in their 20 s, 30 s, and 40 s, respectively. The aloeswood water produced according to the example was poured into coffee cups, and the participants drank it at 10-minute intervals. The taste, aroma, and preference were evaluated using a 9-point rating system.

The results are shown in Table 2.

TABLE 2

| | Taste | Aroma | Preference |
|---|---|---|---|
| Example 1 | 8.2 | 8.3 | 8.2 |
| Example 2 | 8.3 | 8.4 | 8.4 |
| Example 3 | 8.5 | 8.1 | 8.5 |
| Example 4 | 8.2 | 8.2 | 8.2 |
| Example 5 | 8.3 | 8.5 | 8.1 |
| Example 6 | 8.2 | 8.3 | 8.5 |
| Example 7 | 8.4 | 8.2 | 8.3 |
| Example 8 | 8.5 | 8.4 | 8.4 |
| Example 9 | 8.5 | 8.6 | 8.3 |
| Example 10 | 8.2 | 8.4 | 8.5 |
| Example 11 | 8.4 | 8.3 | 8.3 |
| Example 12 | 8.3 | 8.4 | 8.4 |
| Example 13 | 8.5 | 8.2 | 8.6 |
| Example 14 | 8.2 | 8.1 | 8.2 |
| Example 15 | 8.4 | 8.5 | 8.1 |
| Example 16 | 8.3 | 8.2 | 8.4 |
| Example 17 | 8.5 | 8.5 | 8.2 |
| Example 18 | 8.3 | 8.3 | 8.4 |
| Example 19 | 8.5 | 8.3 | 8.5 |
| Example 20 | 8.2 | 8.0 | 8.3 |

As shown in Table 2, it was found that examples prepared by using a method of preparing aloeswood water containing aloeswood showed high taste, aroma, and overall preference scores.

The method of preparing aloeswood water with atopic dermatitis and acne ameliorating effects according to the present invention has the effect of providing a method of preparing aloeswood water by dispersing active ingredients of aloeswood in water, and an easily drinkable aloeswood water prepared by using the method that can provide atopic dermatitis and acne ameliorating effects.

As described above those of ordinary skill in the art to which the present invention pertains can understand that it can be easily modified into other specific forms without changing the technical principles or essential features of the present invention. Therefore, it should be construed that the embodiments described above are illustrative in all respects and not restrictive. The scope of the present invention should be interpreted that all changes or modifications derived from the meaning and scope of the claims described below, rather than the detailed description above, and the equivalent concepts thereof are included in the scope of the present invention.

1. A method of preparing aloeswood water, comprising:
a material preparation step in which materials are prepared by washing each of aloeswood, mulberry Phellinus linteus, Houttuynia cordata Thunb., Saururus chinensis, green tea or puer tea, Stachys sieboldii, Lycopus luci, Agrimonia pilosa, and Panax notoginsengs, and then drying in the shade;
a material pulverization step in which each of the aloeswood, mulberry Phellinus linteus, Houttuynia cordata Thunb., Saururus chinensis, green tea or puer tea, Stachys sieboldii, Lycopus lucidus Turcz., Agrimonia pilosa, and Panax notoginsengs, dried through the material preparation step, is pulverized;
a boiling step in which, after the material pulverization step is completed, aloeswood powder, mulberry Phellinus linteus powder, Houttuynia cordata Thunb. powder, Saururus chinensis powder, green tea powder or puer tea powder, Stachys sieboldii powder, Lycopus lucidus Turcz. powder, Agrimonia pilosa powder, and Panax notoginsengs powder are mixed at a weight ratio of 3:1.3 to 1.7:1.3 to 1.7:1.1 to 1.5:0.8 to 1.2:0.3 to 0.7:0.5 to 0.9:0.1 to 0.5:0.1 to 0.4 to prepare a material mixture, water in an amount of 9,000 to 11,000 parts by weight based on 100 part by weight of the material mixture is mixed, and the resulting mixture is heated and boiled in a temperature range of 80 to 100° C. for 22 to 26 hours;
an infusing step in which, after the boiling step is completed, infusing is performed by keeping the boiled water in a temperature range of 15 to 25° C. for 22 to 26 hours; and
a reduced pressure distillation step in which, after the infusing step is completed, the infused water is distilled under reduced pressure.

2. The method of preparing aloeswood water according to claim 1, wherein, in the reduced pressure distillation step, reduced pressure distillation is performed at a pressure in a range of 0 to 760 mmHg.

3. The method of preparing aloeswood water according to claim 1, wherein the aloeswood in the material preparation step is aloeswood produced by hardening a resin on an aloeswood tree.

4. The method of preparing aloeswood water according to claim 1, wherein the water in the boiling step includes purified water containing minerals that help alcohol decomposition or human body metabolism.
